(19) United States
(12) Patent Application Publication
(10) Pub. No.: US 2018/0125374 A1
(43) Pub. Date: May 10, 2018

(54) Breath Analysis Apparatus and Methods of Use Thereof

(71) Applicants: David R. Hall, Provo, UT (US); Dan Allen, Springville, UT (US); Ben Swenson, Lehi, UT (US); Steven Butala, Provo, UT (US); Terrece Pearman, Draper, UT (US)

(72) Inventors: David R. Hall, Provo, UT (US); Dan Allen, Springville, UT (US); Ben Swenson, Lehi, UT (US); Steven Butala, Provo, UT (US); Terrece Pearman, Draper, UT (US)

(21) Appl. No.: 15/346,037

(22) Filed: Nov. 8, 2016

(51) Int. Cl. A61B 5/0205; G01N 33/497; G01N 1/22; A61B 5/08

(52) U.S. Cl. CPC A61B 5/0205; G01N 33/497; G01N 2001/2244; A61B 5/082; G01N 2033/4975; G01N 1/2205

(57) ABSTRACT

We disclose a device for measuring volatile organic compounds (VOCs) in exhaled breath. The device includes a cover which may be a mask. The cover fits over a user's face as the user breathes. The cover includes one or more removably attached filters which collect VOCs. Some embodiments include reference filters which measure VOCs in the air the user inhales. Reference filter measurements may be subtracted from total VOCs collected from exhaled breath to exclude VOCs that did not originate in the user's body. The filters may be removed from the device and sent for analysis by a gas analyzer which may be a gas chromatograph. Methods of using the device are also disclosed. The device may be used to assess a user's health status, identify or quantify compounds to which a user may have been exposed, or to aid in medical diagnosis.

### BACKGROUND

### Field of the Invention

This invention relates to medical devices for use in measuring the chemical makeup of exhaled breath, and, in particular, measuring exhaled volatile organic compounds.

### Background of the Invention

Breath analysis is useful for detection of a variety of health conditions. However, collecting breath samples is a challenge. Several methods have been used to collect breath samples for analysis. These methods measure volatile organic compounds (VOCs) in a patient's exhaled breath.

In some techniques, a user breathes into a tube which passes through a cryofocusing unit. The cryofocusing unit concentrates the VOCs in the user's exhaled breath. This method is inconvenient due to the required equipment, although results tend to be more quantitative than results from other known methods. After collection, a container with the concentrated VOCs is sent to a lab for analysis using techniques that may include gas chromatograph (GC), gas chromatography mass spectrometry (GC-MS), 2D GC-MS (GC-MS-MS), or GC ion mobility spectrometry GC-IMS.

Some known techniques require the user to breath into a device which performs the collection and the analysis. This method monopolizes the instrument while the breath is being collected.

In addition to the inconvenience of available methods of collecting VOCs from breath, the accuracy of the known methods is somewhat compromised. This is at least because they do not account for VOCs in the environment which the user may breathe in, but which do not have their origin in the body. Consequently, a convenient and accurate way to collect exhaled VOCs for analysis is needed.

### SUMMARY

We disclose a novel device for measuring volatile organic compounds (VOCs) in exhaled breath. The device includes a cover, which may be a mask, that fits over a part of a user's face. The cover may fit over the user's mouth, nose, or both. One or more VOC collecting filters are connected to the cover. The VOC collecting filters collect VOCs from the user's exhaled breath. Various embodiments of the VOC collecting filters are described herein. In some embodiments, the VOC collecting filter(s) is attached to the cover on an inner side of the cover which is adjacent to the user. In other embodiments, the VOC collecting filter(s) is attached to the outer side of the cover which faces away from the user when the device is in use. In some embodiments, the VOC collecting filter(s) is contained within a cartridge, the cartridge including one or more one-way valves.

The device may include a reference filter which collects background VOCs from the environment. This provides a measurement of the VOCs already in the air that the user inhales. This measurement may be subtracted from the measurement of VOCs present in the user's exhaled air so that the final calculated measurement represents only VOCs that were collected from the user's body.

Methods of using the device to collect VOC measurements and obtain an analysis are also disclosed. The results of this analysis may be used to provide an assessment of a user's health, identify and/or quantify a substance to which a user may have been exposed, or to aid in medical diagnosis.

### BRIEF DESCRIPTION OF THE DRAWINGS

FIG. 1A is a drawing of a mask with a VOC collection filter on the inside surface of the mask.

FIG. 1B is a drawing of a mask with a VOC collection filter on the outside surface of the mask.

FIG. 2 is a drawing of an embodiment of a VOC collection filter within an envelope which may be attached to a mask using slots in the mask.

FIG. 3A is a drawing of an embodiment of a VOC collection filter showing perforations for removing the VOC collection filter.

FIG. 3B is a side view of a containing a VOC collection filter within a protective film.

FIG. 4 is a drawing of an embodiment of the device with a single cartridge.

FIG. 5 is a drawing of an embodiment of the device with two cartridges.

FIG. 6A is an illustration of a cartridge for collecting VOCs from exhaled air with no air moving through it.

FIG. 6B is an illustration of a cartridge for collecting VOCs from exhaled air with exhaled air moving through it.

FIG. 6C is an illustration of a cartridge for collecting background VOCs with no air moving through it.

FIG. 6D is an illustration of a cartridge for collecting background VOCs with room air moving through it.

FIG. 7 is a drawing of a user wearing an embodiment of the disclosed device.

FIG. 8 is a flow chart describing a method of using an embodiment of the invention.

### DETAILED DESCRIPTION

### Definitions

Mask, as used herein, means a cover that may be placed over a user's face.

Air, as used herein, means a gas or gas mixture that may be breathed in by a human or animal, including room air, breathable gases supplied by a gas source, and air that has been exhaled by a human or animal after being inhaled.

User, as used herein, means a human or animal capable of using the disclosed device.

Outer side, as used herein and in reference to the cover, means the side that is furthest from the user when the device is in use.

Inner side, as used herein and in reference to the cover, means the side that is nearest the user when the device is in use.

Disclosed herein is a device that includes a cover that may be placed over a user's nose, mouth, or both the nose and mouth, and which collects volatile organic compounds (VOCs) that may be present in a user's exhaled breath. In some embodiments, the cover is a mask. The cover includes one or more VOC collection filters (hereinafter, “filter” or “filters” or “filter(s)”) which may be removed from the cover and inserted into a gas analyzer or other device to measure the VOCs caught in the filters. The measurements may be used to assess the user's health status, identify or quantify a compound to which the user may have been exposed, or aid in making a medical diagnosis. For example, the user may have been exposed to a potentially toxic compound that is exhaled or that is metabolized to a byproduct that is exhaled. In another example, a user may be suspected of suffering from a disease that causes the user to exhale a certain compound. In yet another example, the composition of the user's exhaled breath may give an indication of the user's physiological status.

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

According to an embodiment of the invention, one or more filters may be attached to a cover. The cover may be a mask worn on a user's face covering the mouth, nose, or mouth and nose. The filter(s) may be attached to the cover using a variety of attachment methods and may be positioned on the cover in a variety of configurations. The filter(s) may comprise a material that is air permeable. The cover may comprise of an area constructed from material that is relatively impermeable to air and an area constructed from material that is permeable to air. The filter(s) may be placed on or adjacent to the area that is permeable to air so that air crosses the cover through the filter(s).

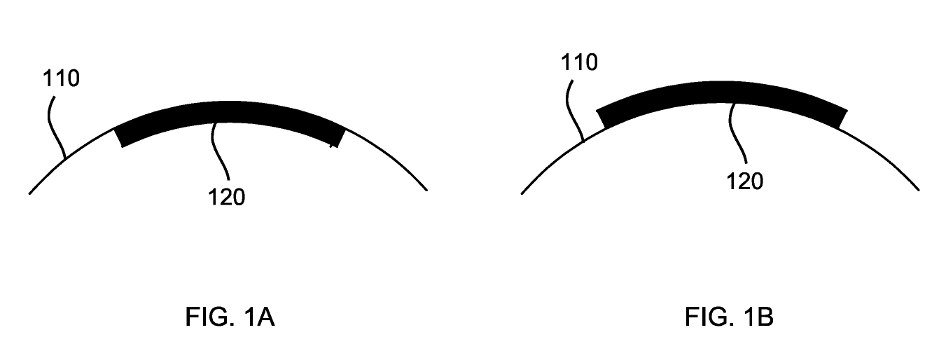

FIG. 1A illustrates mask 110 which includes filter 120. In this embodiment, filter 120 is attached to mask 110 on the inner surface of mask 110 which is the surface nearest the user's face when in use. In contrast, FIG. 1B illustrates mask 110 with filter 120 attached to the outer surface of mask 110 relative to the user's face when the device is in use. In the embodiment of either FIG. 1A or 1B, the part of mask 110 over which filter 120 is positioned may be more air permeable than the rest of mask 110 so air may flow into filter 120.

The filter may be removably attached to the device using a variety of attachment methods. These include, but are not limited to, perforations which may be torn to remove the filter, nonpermanent adhesive, tape, clip(s), brad(s), tab(s), grommet(s), pin(s), and slot(s).

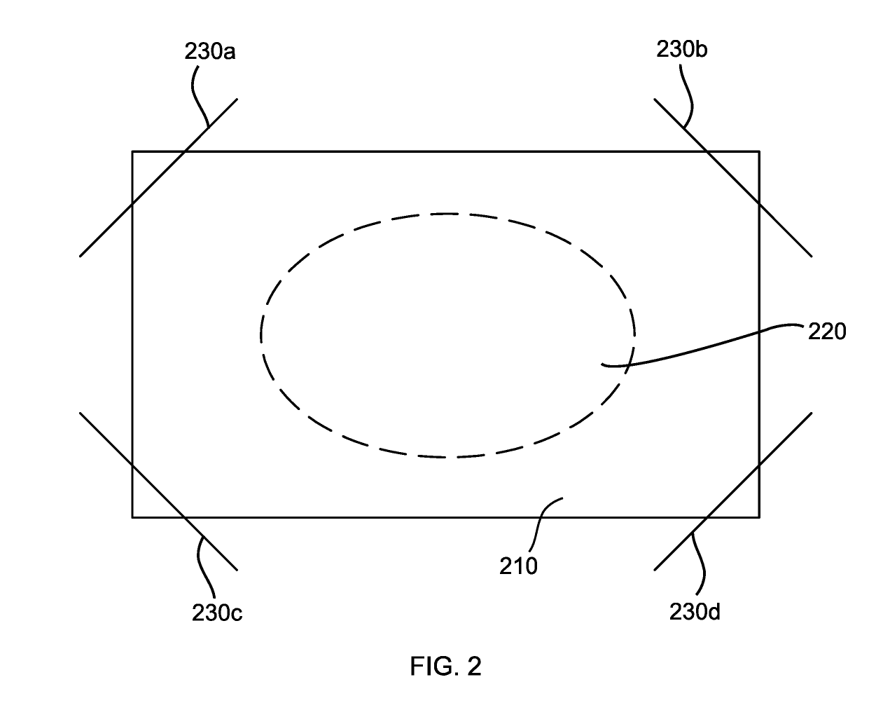

FIG. 2 illustrates an embodiment of a filter in which filter 210 is removably attached to a mask by inserting the corners of filter 210 into slots 230a, 230b, 230c, and 230d. Area 220, which is surrounded by a dashed line in FIG. 2, illustrates the area through which most of the air passes for VOC collection. Slots 230a-230d are located on the mask. In this embodiment, a new filter 210 may be quickly and easily slipped into the slots of a mask prior to use. Then filter 210 may be slipped out and sent to be analyzed for collected VOCs.

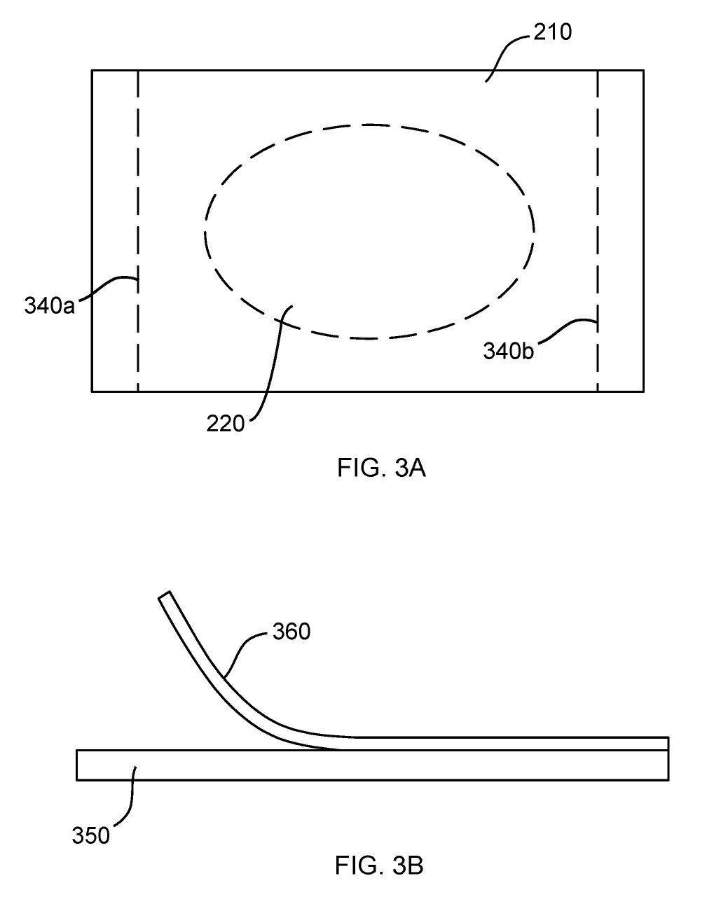

FIG. 3A illustrates filter 210 which further includes perforations 340a and 340b. In this embodiment, the ends of filter 210 may be torn or otherwise severed along perforations 340a and 340b to release the center area of filter 210. This center area includes area 220 where air most of the air crosses filter 210 and most of the VOCs are collected. The center area of filter 210 may then be analyzed for VOC content.

FIG. 3B illustrates an embodiment of a filter that includes protective film 360 which may cover filter 350. Protective film 360 may cover filter 350 or other embodiments thereof so that the filter does not collect VOCs from ambient air prior to use. In other embodiments, protective covering other than film may be used.

In FIG. 3B, protective film 360 is shown as it is being peeled back from filter 350. Filter 350, or other embodiments thereof, may then be attached to the mask as described above. With protective film 360 removed, filter 350 is exposed to air. Filter 350 is thus able to collect VOCs from either exhaled air or the air available for the user to inhale. After use, filter 350, and other embodiments of the filter, may be placed in an air tight container after use so that no more VOCs are collected prior to analysis.

In some embodiments, the filter may be within a cartridge. The cartridge may contain a first one-way valve or the mask may contain a one-way valve in a mounting fixture where the cartridge inserts into or onto the mask. The cartridge or mounting fixture may also include a second one-way valve. The two one-way valves may be located on opposite sides of the filter. In some embodiments, both one-way valves are within the cartridge, in some embodiments, both one-way valves are in the mounting fixture, and in some embodiments one of the first one-way valve is in the cartridge and the second one-way valve is in the mounting fixture. In embodiments which include a first and a second one-way valve, the two one-way valves define a volume between them that creates an isolated space within the cartridges when there is not sufficient air pressure applied on the valves to open them.

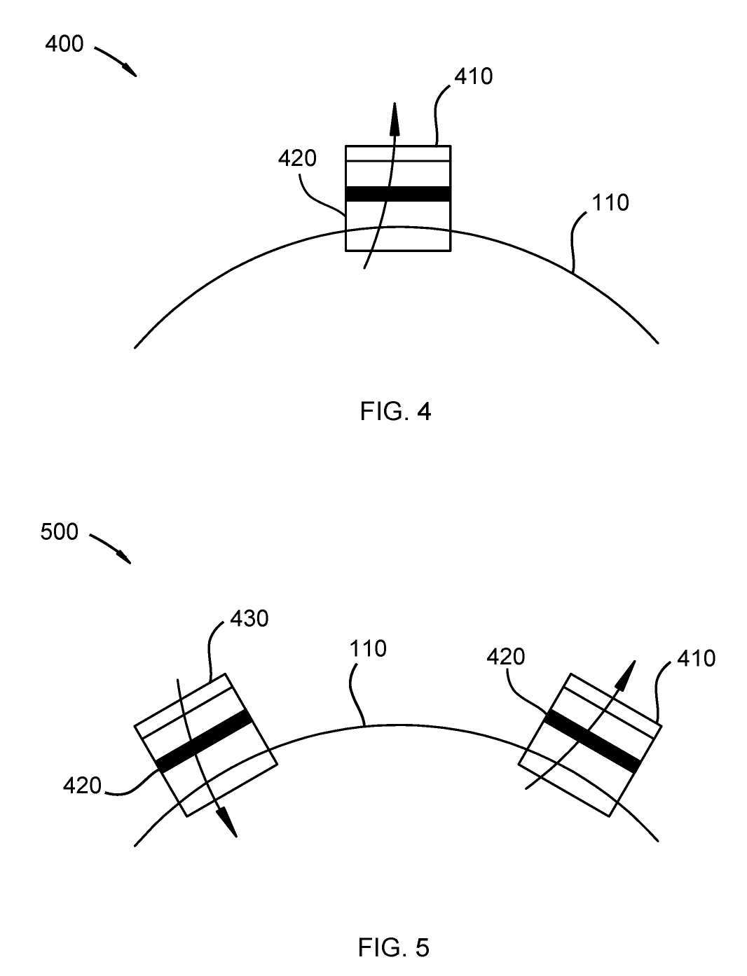

FIG. 4 illustrates a section of device 400 which is an embodiment of the invention disclosed herein. Device 400 includes mask 110 and cartridge 410. Cartridge 410 includes filter 420. Mask 110 is shown concave and may comprise of a polymer or copolymer which is impermeable to air. The section of the mask covered by the cartridge may be permeable to air so that air flows through the cartridge. The arrow shows the direction of air flow through cartridge 410. Specifically, exhaled air travels from the inner side of the mask, through cartridge 410, to the outside of mask 110. In this embodiment, filter 420 collects VOCs from the user's exhaled air.

FIG. 5 illustrates a section of device 500. Device 500 is similar to device 400 of FIG. 4. However, in addition to cartridge 410, device 500 includes a second cartridge, shown as cartridge 430. Like cartridge 410, cartridge 430 includes filter 420. In some embodiments, the filters in the two cartridges may comprise of the same materials and/or configurations and in other embodiments they may comprise of different materials and/or configurations. As in device 400, exhaled air passes to the outside of mask 110 through cartridge 410 of device 500. Cartridge 430 of device 500 provides a reference filter. Air from outside of device 500 travels inward from the outside of mask 110 to the inside of mask 110. VOCs in air to be inhaled or simply a sample of the ambient air may be collected by filter 420 in cartridge 430. In some embodiments, the air to be inhaled is room air. In other embodiments, it is air provided from a gas source and transmitted to the area between the device and the user's face so that the user may inhale the air. Measurements from the reference filter provide background VOC measurements to compare to the VOCs measured in the user's exhaled air. A more accurate measurement of how much VOCs are produced by the user's body may be obtained by subtracting the VOCs measured in the inhaled air source from the VOCs measured in the user's exhaled breath.

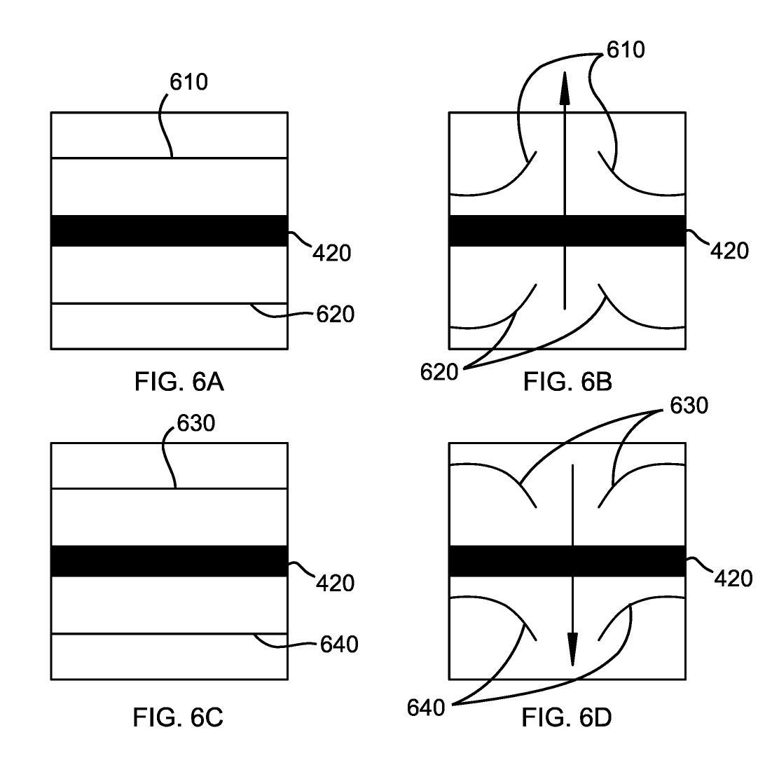

FIGS. 6A and 6B are close-up illustrations of an embodiment of cartridge 410. FIGS. 6A and 6B show a first one-way valve, valve 610, and a second one-way valve, valve 620. Filter 420 is positioned between valve 610 and valve 620. FIG. 6A shows valves 610 and 620 in a resting position. In this state, there is not sufficient air pressure applied on the valves to open them. This creates an isolated space within the cartridge and no air passes through cartridge 410.

FIG. 6B illustrates cartridge 410 in a state in which enough air pressure is applied to valves 610 and 620 to open the valves. This may be a situation in which a user has exhaled into a device as disclosed herein causing the exhaled air to exit the device through cartridge 410. The arrow illustrates the direction of air flow which is outward to the outside environment. Note that valves 610 and 620 are one-way valves which open in the direction shown by the arrow in FIG. 6B. Therefore, should the user inhale, air may not be drawn through cartridge 410 in a direction that is opposite that shown in FIG. 6B and VOCs from air not yet inhaled by the user will not be measured by filter 420 in cartridge 410.

FIGS. 6C and 6D are close-up illustrations of an embodiment of cartridge 430. FIGS. 6C and 6D show a third one-way valve, valve 630, and a fourth one-way valve, valve 640. Filter 420 is positioned between valve 630 and valve 640. FIG. 6C shows valves 630 and 640 in a resting position. In this state, there is not sufficient air pressure applied on the valves to open them. This creates an isolated space within the cartridge and no air passes through cartridge 430.

FIG. 6D illustrates cartridge 430 (as first shown in FIG. 5) in a state in which enough air pressure is applied to valves 630 and 640 to open the valves. This may be a situation in which a user has inhaled thereby causing the air to enter the device through cartridge 430. The arrow illustrates the direction of air flow which is inward from the outside environment to the inner side of the mask that is nearest the user. Note that valves 630 and 640 are one-way valves which open in the direction shown in FIG. 6D. Therefore, should the user exhale, air may not be drawn through cartridge 420 in a direction that is opposite that shown by the arrow in FIG. 6B and VOCs from exhaled air will not be measured by filter 420 in cartridge 430. In this situation, cartridge 430 contains a reference filter as discussed herein.

In some embodiments, the user inhales room air. In embodiments which include a reference filter, the room air flows through the reference filter to the space behind the cover to be inhaled by the user. In other embodiments, air provided by a gas source may be directed into cartridge 430. For example, one end of a section of tubing may be connected to a source of isolated air and the other end of the tubing may be connected to an air port in the device. In some embodiments, the air port extends from the cartridge that houses the reference filter so that the air is directed into the reference filter.

The device may be part of a biomedical sensing station. The biomedical sensing station may include a gas source for inhaled air. It may also include other devices for conducting measurements that may be relevant to a user's health status. In some embodiments, the biomedical sensing station is a medical toilet which may measure blood pressure, breathing rate, body weight, collect electrocardiogram measurements, conduct laboratory analyses on bodily waste, and other perform measurements that may be extrapolated to physiological functions.

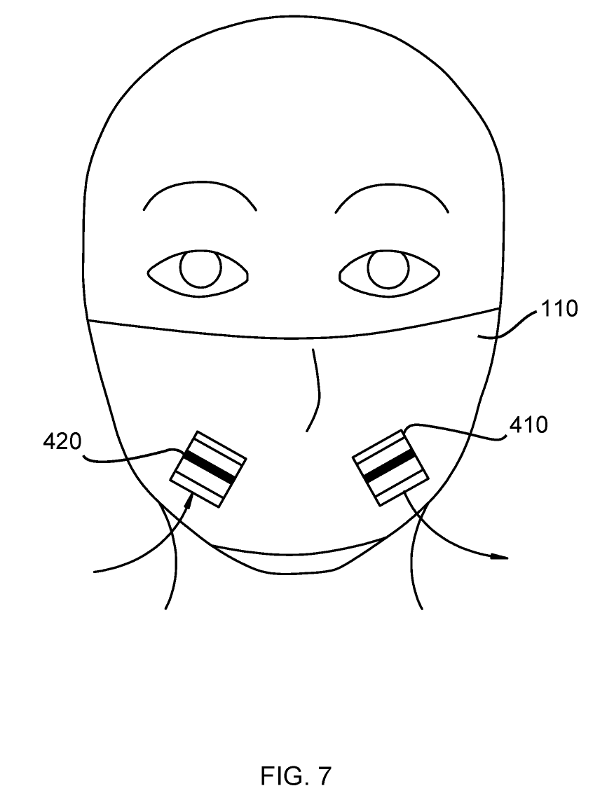

FIG. 7 illustrates an embodiment of the device during use. The embodiment of FIG. 7 is similar to that of FIG. 5. Inhaled air travels through cartridge 420 as shown by the arrow which indicates the direction of air flow. The filter within cartridge 420 acts as a reference filter and measures background VOCs in the air the user will inhale. In contrast, exhaled air travels from the inside of mask 110 through cartridge 410 as shown by the arrow which indicates the direction of air flow. The filter within cartridge 410 collects exhaled VOCs.

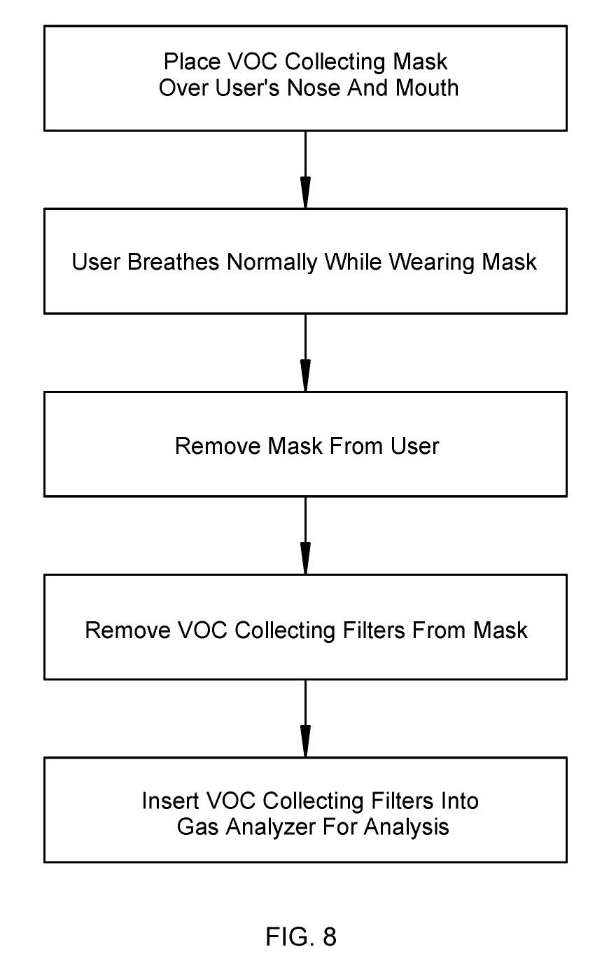

FIG. 8 provides a flow chart illustrating an embodiment of a method of using the disclosed device. The device is placed adjacent to a user's face. In the embodiment of FIG. 8, the mask is placed over a user's mouth and nose. However, in some embodiments the mask may cover only the user's mouth or only the user's nose. The user breathes while wearing the mask. The filters in the mask collect exhaled VOCs while the user breathes. The mask is then removed from the user's face and the filters are removed from the cartridges. At this point the filters may be directly inserted into a gas analyzer or other device that measures VOCs. Alternatively, the filters may be placed in air tight containers and analyzed later.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A device for collecting volatile organic compounds for analysis from the breath of a user comprising:
a concave mask, the mask comprising:
an outer side;
an inner side;
an air permeable area; and
an air impermeable area, the air impermeable area comprising a polymer or copolymer;
a first cartridge;
wherein the first cartridge is removably attached to the mask,
wherein the first cartridge is positioned adjacent to the air permeable area of the mask,
wherein air flows from the inner side to the outer side of the mask through the first cartridge, and
wherein the first cartridge comprises:
a first one-way valve; and
a first filter;
wherein the first filter comprises a material which collects volatile organic compounds,
wherein the first filter is removably positioned within the first cartridge, and
wherein the first filter is air permeable.

2. The device of claim 1, wherein the first filter comprises one or more of the following: film, carbon paper, polymer, metal frit, molecular sieve, and carbon cloth.

3. The device of claim 1, wherein the first cartridge is attached to a first mounting fixture, wherein the first mounting fixture is attached to the outer side of the mask.

4. The device of claim 1, wherein the first cartridge further comprises a second one-way valve, and where in the first filter is positioned between the first one-way valve and the second one-way valve.

5. The device of claim 7, wherein the first and second one-way valves permit air flow from the outer side to the inner side of the mask.

6. The device of claim 1, wherein the mask further comprises an air port.

7. The device of claim 1, wherein the device is incorporated into a biomedical sensing station.

8. The device of claim 7, wherein the biomedical sensing station is a medical toilet.

9. The device of claim 8, wherein the medical toilet measures blood pressure and analyzes the content of urine and feces.

10. The device of claim 1 further comprising:
a second cartridge;
wherein the second cartridge is removably attached to the mask,
wherein the second cartridge is positioned adjacent to the air permeable area of the mask,
wherein air flows from the outer side to the inner side of the mask through the second cartridge and
wherein the second cartridge comprises:
a third one-way valve; and
a second filter;
wherein the second filter comprises a material which collects volatile organic compounds,
wherein the second filter is removably positioned within the second cartridge, and
wherein the second filter is air permeable.

11. The device of claim 10, wherein the second filter comprises one or more of the following: film, carbon paper, polymer, metal frit, molecular sieve, and carbon cloth.

12. The device of claim 10, wherein the second cartridge is attached to a second mounting fixture, wherein the second mounting fixture is attached to the outer side of the mask.

13. The device of claim 10, wherein the second cartridge further comprises a fourth one-way valve, and where in the second filter is positioned between the third one-way valve and the fourth one-way valve.

14. The device of claim 13, wherein the third and fourth one-way valves permit air flow from the inner side to the outer side of the mask.

15. The device of claim 10, wherein the first filter accumulates volatile organic compounds from an air source air to be inhaled by a user and the second filter accumulates volatile organic compounds from air exhaled by a user.

16. The device of claim 10, wherein the mask further comprises an air port.

17. The device of claim 10, wherein the device is incorporated into a biomedical sensing station.

18. The device of claim 10, wherein the biomedical sensing station is a medical toilet.

19. The device of claim 18, wherein the medical toilet measures blood pressure and analyzes the content of urine and feces.

20. The device of claim 19, wherein the medical toilet comprises a container which houses breathable air, wherein second cartridge is connected to an air port, and wherein the air port is connected to the container.
